(19) United States
(12) Patent Application Publication
(10) Pub. No.: US 2018/0327819 A1
(43) Pub. Date: Nov. 15, 2018

(54) BIOSENSOR COMPRISING TANDEM REACTIONS OF STRUCTURE SWITCHING, NUCLEOLYTIC DIGESTION AND AMPLIFICATION OF A NUCLEIC ACID ASSEMBLY

(71) Applicant: McMaster University, Hamilton (CA)

(72) Inventors: Yingfu Li, Dundas (CA); John Brennan, Dundas (CA); Meng Liu, Dundas (CA)

(21) Appl. No.: 15/738,821

(22) PCT Filed: Jun. 22, 2016
(86) PCT No.: PCT/CA2016/050731

§ 371 Date: Dec. 21, 2017

Related U.S. Application Data
(60) Provisional application No. 62/182,711, filed on Jun. 22, 2015.

(51) Int. Cl. C12Q 1/6825; C12Q 1/6853; G01N 33/58

(52) U.S. Cl. CPC C12Q 1/6825; C12Q 1/6853; C12Q 2525/307; C12Q 2525/205; C12Q 2531/125; G01N 33/582

(57) ABSTRACT

The present application relates to a biosensor for detecting analytes, various kits and methods of use thereof. In particular, the biosensor's mode of operation is based on binding of analytes to a nucleic acid sequence which triggers rolling circle amplification and detection of the amplified product as the indicator of the presence of the analytes.

The present application claims the benefit of priority from U.S. provisional patent application No. 62/182,711, filed Jun. 22, 2015, the contents of which are incorporated herein by reference.

### FIELD

The present application relates to biosensors for detecting analytes, various kits and methods of use thereof. In particular the biosensor comprises rolling circle amplification (RCA) templates in which primer activation and amplification reactions are triggered by binding of an analyte.

### BACKGROUND

DNA amplification is a valuable tool in genomics, molecular diagnosis, chemical biology, and DNA nanotechnology. In addition to polymerase chain reaction,^{[1]} an isothermal DNA amplification technique known as “rolling circle amplification” (RCA) has recently attracted great attention.^{[2,3]} RCA involves elongation of a DNA primer over a circular DNA template by DNA polymerases with strand-displacement ability and high processivity, such as φ29 DNA polymerase (ϕ29DP).^{[4]} These polymerases can continuously dislodge newly synthesized DNA strands from the circular template, making it available for many rounds of copying. The product of RCA is extremely long single-stranded (ss) DNAs with thousands of repeating units.^{[2,3]} Due to its amplification power and operational simplicity, RCA has become a popular DNA amplification technique.^{[5-9]}

Nature has evolved DNA polymerases into impressive enzymes with multiple functions. For example, φ9DP is capable of carrying out 3′-5′ exonucleolytic digestion of ssDNAs (but not double-stranded DNAs),^{[10]} in addition to its DNA polymerization and strand-displacement functions.^{[4]} The nucleolytic activity, common among DNA polymerases, has been evolved to proofread DNA replication in vivo.^{[11]} However, this property is rarely explored for in vitro applications.

### SUMMARY

The present application demonstrates a versatile amplified biosensing strategy which uniquely integrates Rolling Circle Amplification (RCA), structure-switching nucleic acid molecules for target recognition and exonucleolytic trimming and nucleic acid-dependent polymerization functions of a nucleic acid polymerase which features a two-duplex tripartite nucleic acid assembly. The biosensing strategy of the present application is capable of delivering a limit of detection that is several orders of magnitude lower than the dissociation constant of the structure-switching nucleic acid molecules that binds its corresponding analyte.

Accordingly, the present application includes a biosensor for detecting an analyte comprising a nucleic acid assembly wherein the nucleic acid assembly comprises:
(a) a circular single-stranded nucleic acid molecule that is a rolling circle amplification (RCA) template;
(b) a linear single-stranded nucleic acid molecule that binds the analyte; and
(c) a linear single-stranded nucleic acid molecule comprising a first nucleic acid sequence that is a primer for the RCA template and a second nucleic acid sequence that is digested by a nucleic acid polymerase having exonuclease activity,
   wherein the first nucleic acid sequence of the linear single-stranded nucleic acid molecule binds to a portion of the circular single-stranded nucleic acid molecule and the second nucleic acid sequence of the linear single-stranded nucleic acid molecule binds to a portion of the analyte-binding single-stranded nucleic acid molecule in the absence of the analyte and in the presence of the analyte the binding of the second nucleic acid sequence of the linear single-stranded nucleic acid molecule to the portion of the analyte-binding single-stranded nucleic acid molecule is disrupted making the second nucleic acid sequence available for digestion by the nucleic acid polymerase having exonuclease activity.

The present application also includes assay methods that utilize the biosensor of the present application. In some embodiments, the assay is a method of detecting an analyte in a sample, wherein the sample is suspected of comprising the analyte, the method comprising contacting the sample with the biosensor of the present application, and monitoring for a presence of a nucleic acid product from the RCA template wherein the presence of the nucleic acid product from the RCA template indicates the presence of the analyte in the sample.

The present application further includes kits comprising the biosensors of the application. In some embodiments, the kit includes the biosensor and any further reagents for performing an assay using the biosensor, for example a nucleic acid polymerase having exonuclease activity. In some embodiments, the kit includes instructions for using the biosensor in the assay and any controls needed to perform the assay. The controls may be on the biosensor itself, or alternatively, on a separate substrate. In some embodiments, the kit includes all the components required to perform any of the assay methods of the present application.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 (A) shows a schematic representation of one embodiment of a biosensor of the application, and (B)-(D) show the digestion of one exemplary biosensor comprising 1 μM radioactive PP1 with 0.1 U/μL ϕ29DP analyzed by 20% dPAGE wherein (B) represents PP1 alone for 0-60 min, (C) in the presence of 0-2.5 μM AP1 for 30 min, and (D) in the presence of 1.5 μM AP1 and 0.5 mM ATP.

FIG. 2 shows digestion of one exemplary biosensor of the application comprising 1 μM radioactive PP1 with 0.1 U/μL ϕ29DP for 30 min in the presence of (A) 0-2.5 μM CT1, (B) 1 μM CT1, 1.5 μM AP1, 0.5 mM ATP, and (C) 1 μM CT1, 1.5 μM AP1, 0.5 mM GTP.

FIG. 3 shows an agarose gel analysis of an exemplary biosensor of the application wherein RCA products (RP) from RCA reactions of PP1, CT1 and AP1 in the absence (A) and presence (B) of ATP.

FIG. 4 shows (A) digestion of 1 μM radioactive PP2 with 0.1 U/μL ϕ29DP for 30 min in the presence of 1 μM CT1, 1.5 μM I-AP2 and 100 nM PDGF, (B) and (C) agarose gel analysis of RP in RCA reaction mixtures containing various combinations of 1 μM PP2, 1 μM CT1, and 1.5 μM I-AP2 in the absence and presence of 100 nM PDGF of an exemplary biosensor of the application.

FIG. 5 shows detection of PDGF using an exemplary biosensor of the application: (A) agarose gel analysis of RP in RCA reaction mixtures containing 1 μM PP2, 1 μM CT1, 1.5 μM I-AP2, and increasing concentrations of PDGF, (B) working principle of hyper-branched RCA (HRCA), (C) real-time fluorescence monitoring of HRCA reaction with EvaGreen, wherein the concentrations are represented as 0 (baseline), 1 fM (second line from the x-axis), 10 fM (third line from the x-axis), 100 fM (fourth line from the x-axis), 1 pM (fifth line from the x-axis), 10 pM (sixth line from the x-axis), 100 pM (seventh line from the x-axis), 1 nM (top line), and (D) fluorescence readings at 120 min as a function of PDGF concentration.

FIG. 6 shows nucleolytic digestion of one embodiment of a biosensor of the application comprising 5′-FAM labeled AP1 by ϕ29DP in the PP1-AP1 hybrid. Each reaction was performed for 60 min at 30° C. in 50 μL o 1×RCA reaction buffer containing 1 μM AP1, 0.1 U/μL ϕ29DP and varying concentrations of PP1. The reaction mixtures were analyzed by 20% dPAGE.

FIG. 7 shows the effect of GTP on PP1 degradation in the exemplary biosensor, PP1-AP1 hybrid. The experiment was performed for 30 min at 30° C. in 50 μL of 1×RCA reaction buffer containing 1 μM PP, 1.5 μM AP1, 0.1 U/μL ϕ29DP and 0.5 mM GTP. The reaction mixtures were analyzed by 20% dPAGE

FIG. 8 shows (A) digestion of an exemplary biosensor comprising 1 μM radioactive I-PP1 with 0.1 U/μL ϕ29DP for 30 min in the presence of 0-2.5 μM CT1 with the reaction mixtures analyzed by 20% dPAGE, (B) 0.6% agarose gel analysis of RP in RCA reaction mixtures containing PP1-CT1 or I-PP1-CT1 (I: an inverted dT at the 3′-end of PP1 (dot in the graphics)).

FIG. 9 shows a specificity test using an exemplary biosensor of the application for PDGF. (A) RCA reactions with I-AP2M, a mutant aptamer probe (see Table 1 for its sequence). The target binding reaction was first carried out for 30 min at RT in 50 μL of 1×RCA reaction buffer containing 1 μM PP2, 1.5 μM I-AP2M, 1 μM CT1, 100 nM PDGF, or a combination of these as shown. The RCA reaction was then initiated by the addition of 5 U DNAP, 0.4 mM dNTPs, followed by incubating at 30° C. for 1 h. (B) RCA reaction with various protein targets. The target binding reaction was first carried out for 30 min at RT in 50 μL of 1×RCA reaction buffer containing 1 μM PP2, 1.5 μM I-AP2, 1 μM CT1 and 100 nM BSA, thrombin, IgG or PDGF. The reaction mixtures were analyzed by 0.6% agarose gel.

FIG. 10 shows digestion of an exemplary biosensor of the application comprising 1 μM radioactive PP3 with 0.1 U/μL ϕ29DP for 30 min in the presence of 1 μM CT1, 1.5 μM I-DP1 and 100 nM HCV-1 DNA. The reaction mixtures were analyzed by 20% dPAGE.

FIG. 11 shows DNA detection of an exemplary biosensor of the application. (A) shows the EvaGreen-assisted fluorescence monitoring of HRCA reaction for the detection of HCV-1 at concentrations of 0.2 pM (fifth line from the x-axis), 2 pM (sixth line from the x-axis), 20 pM (seventh line from the x-axis), 0.2 nM (eighth line from the x-axis), 2 nM (ninth line from the x-axis) and 20 nM (top line), (B) shows the EvaGreen-assisted fluorescence monitoring of HRCA reaction for the detection of HCV-1 at concentrations of 0 aM (first line from the x-axis), 20 aM (second line from the x-axis), 0.2 fM (third line from the x-axis), 2 fM (fourth line from the x-axis) and 20 fM (top line), (C) shows the fluorescence readings at 180 min of HRCA reactions with 0.02-200 fM HCV-1 as a function of HCV-1 concentration, and (D) shows the specificity test of HCV-M1 (first line from the x-axis), HCV-M2 (second line from the x-axis) and HCV-1 (top line).

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “analyte” as used herein means any agent for which one would like to sense or detect using a biosensor of the present application. The term analyte also includes mixtures of compounds or agents such as, but not limited to, combinatorial libraries and samples from an organism or a natural environment.

The term “sample(s)” as used herein refers to any material that one wishes to assay using the biosensor of the application. The sample may be from any source, for example, any biological (for example human or animal medical samples), environmental (for example water or soil) or natural (for example plants) source, or from any manufactured or synthetic source (for example food or drinks). The sample is one that comprises or is suspected of comprising one or more analytes.

The term “nucleic acid” refers to polynucleotides such as deoxyribonucleic acid (DNA) and ribonucleic acid (RNA).

The term “aptamer” as used herein refers to short, chemically synthesized, single stranded (ss) RNA or DNA oligonucleotides which fold into specific three-dimensional (3D) structures that bind to a specific analyte with dissociation constants, for example, in the pico- to nano-molar range.

The term “rolling circle amplification” or “RCA” as used herein refers to a unidirectional nucleic acid replication that can rapidly synthesize multiple copies of circular molecules of DNA or RNA. The term RCA also includes “hyper-branched rolling circle amplification” or “HRCA” which is a technique derived from rolling circle amplification to improve upon the sensitivity of RCA by using both forward and reverse primers.

The term “exonucleolytic trimming” or “exonucleolytic digestion” as used herein refers to the cleaving of nucleotides one at a time from the end (exo) of a polynucleotide chain by a nucleic acid exonuclease.

The term “gel electrophoresis” or “electrophoresis system” as used herein refers to a technique to separate biological macromolecules including proteins or nucleic acids (nucleic acid electrophoresis), according to their electrophoretic mobility. The gel electrophoresis process can be performed under denaturing or non-denaturing conditions.

As used herein in this specification and the appended claims, the singular forms “a”, “an” and “the” include plural references unless the content clearly dictates otherwise. Thus for example, a composition containing “an analyte” includes one such analyte or a mixture of two or more analytes.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific manipulation to be performed, and the identity of the molecule(s) to be transformed, but the selection would be well within the skill of a person trained in the art.

### II. Biosensors of the Application

A versatile amplified biosensing strategy has been demonstrated in the present application which uniquely integrates structure-switching nucleic acid sequences for target recognition with both exonucleolytic trimming and DNA-dependent polymerization functions of a nucleic acid polymerase, such as ϕ29DP. The biosensor features a two-duplex tripartite DNA assembly comprising a circular DNA template, a pre-primer and an analyte binding/recognition sequence. A target-induced analyte binding/recognition sequence structure-switching event acts as the control element for the trimming event carried out by the nucleic acid polymerase, which in turn controls the amplification event executed also by the nucleic acid polymerase. To the best of the Applicant's knowledge, the integrated recognition-digestion-amplification strategy has never been previously reported. Furthermore, this approach can be adopted for detection of a wide-range of targets, including small molecules, proteins and DNA. With incorporation of HRCA, the biosensing strategy of this present application is capable of delivering a limit of detection that is several orders of magnitude lower than the dissociation constant of the aptamer, for example as low as attomolar concentrations of analyte are detected. Therefore, this approach can turn an analyte binding/recognition sequence with a relatively low affinity for its target into an ultra-sensitive biosensing system. With a wide variety of analyte binding/recognition sequences currently available and new sequences that can be conveniently produced by in vitro selection, it is envisioned that the described strategy will find diverse applications.

Therefore, the present application includes a biosensor enabling analyte-dependent rolling circle amplification (RCA) comprising a nucleic acid assembly consisting of a circular single-stranded nucleic acid sequence (in some embodiments named circular template) that can be used as the template for RCA, a single-stranded nucleic acid sequence (in some embodiments named pre-primer) that can be used as the primer for RCA upon digestion of blocking nucleotides, and an analyte-binding single-stranded nucleic acid sequence (in some embodiments named binding sequence).

In some embodiments, the circular template, pre-primer and binding sequences are all DNA molecules. In some embodiments, circular template, pre-primer and binding sequences are all RNA molecules. In some embodiments, one or more of the circular template, pre-primer and binding sequences are DNA molecules and others are RNA molecules. In some embodiments, the binding sequence is a DNA or RNA aptamer. In some embodiments, the binding sequence is a DNAzyme or ribozyme. In some embodiments, the binding sequence is an antisense sequence of a nucleic acid molecule. In some embodiments, the circular template, pre-primer and binding sequences form an assembly through the formation of nucleic acid duplexes.

In some embodiments, the biosensor of the present application functions according to the following chain of reactions: a) the analyte causes the release of the binding sequence from the pre-primer/circular template/binding sequence assembly; b) the DNA polymerase then converts the pre-primer on the pre-primer/circular template assembly into the mature primer through 3′-5′ exonucleolytic digestion; c) the DNA polymerase then uses the mature primer to copy the circular template, resulting in a long-chain DNA products.

In some embodiments, the long-chain DNA products can be detected by fluorescence, color change or other methods.

The present application also includes a biosensor for detecting an analyte comprising a nucleic acid assembly wherein the nucleic acid assembly comprises:
(a) a circular single-stranded nucleic acid molecule that is a rolling circle amplification (RCA) template;
(b) a linear single-stranded nucleic acid molecule that binds the analyte; and
(c) a linear single-stranded nucleic acid molecule comprising a first nucleic acid sequence that is a primer for the RCA template and a second nucleic acid sequence that is digested by a nucleic acid polymerase having exonuclease activity,
   wherein the first nucleic acid sequence of the linear single-stranded nucleic acid molecule binds to a portion of the circular single-stranded nucleic acid molecule and the second nucleic acid sequence of the linear single-stranded nucleic acid molecule binds to a portion of the analyte-binding single-stranded nucleic acid molecule in the absence of the analyte and in the presence of the analyte the binding of the second nucleic acid sequence of the linear single-stranded nucleic acid molecule to the portion of the analyte-binding single-stranded nucleic acid molecule is disrupted making the second nucleic acid sequence available for digestion by the nucleic acid polymerase having exonuclease activity.

In some embodiments, (a), (b) and (c) are independently selected from DNA molecules and RNA molecules. In some embodiments, (a), (b) and (c) are DNA molecules. In some embodiments, (a), (b) and (c) are RNA molecules. In some embodiments, (a), (b) and (c) comprise a combination of DNA and RNA molecules.

In some embodiments, the circular single-stranded nucleic acid molecule is prepared from a precursor 5′-phosphorylated linear single-stranded nucleic acid molecule through circularization with a T4 nucleic acid ligase and a circularization nucleic acid template. In some embodiments, the precursor 5′-phosphorylated linear single-stranded nucleic acid molecule is ACTGTAACCA TTCTT GTTTC GTATC ATTGC AGAATTCTAC TAATT TATCT GAATACCGTG [SEQ ID NO:1]. In some embodiments, the circular single-stranded nucleic acid molecule that is a rolling circle amplification (RCA) template is GTTAC AGTCA CGGTA T [SEQ ID NO:2].

In some embodiments, the linear single-stranded nucleic acid molecule that binds the analyte, or the binding sequence, is selected from a nucleic acid aptamer, a nucleic acid enzyme and an antisense sequence of a nucleic acid molecule. In some embodiments, the linear single-stranded nucleic acid molecule that binds the analyte is a sequence that is resistant to nuclease digestion. In some embodiments, resistance to nuclease digestion is conferred on a nucleic acid sequence by the presence of a hairpin secondary structure. In some embodiments, the linear single-stranded nucleic acid molecule binds the analyte with specificity. By binding the analyte with specificity it is meant that the linear single-stranded nucleic acid molecule binds only the analyte to be detected, even in the presence of other analytes, at least within the limits of detection of the present sensor.

In some embodiments, the nucleic acid aptamer is a DNA aptamer or an RNA aptamer. In some embodiments, the nucleic acid aptamer is produced using Systematic Evolution of Ligands by Exponential enrichment (SELEX) technology, for example as described in A. D. Ellington and J. W. Szostak, Nature 346(6287), 818-822 (1990). In some embodiments, the nucleic acid aptamer is a DNA aptamer. In some embodiments, the DNA aptamer is

[SEQ ID NO: 7]
CACTG ACCTG GGGGA GTATT GCGGA GGAAGGT.

In some embodiments, the DNA aptamer is

[SEQ ID NO: 8]
CAGGC TACGG CACGT AGAGC ATCAC CATGA TCCTG/3invdT/.

In some embodiments, the DNA aptamer is

[SEQ ID NO: 9]
CAGGC TACGG CACTT TTTTC ATTTAAATTA TAATT/3invdT/.

In some embodiments, the nucleic acid enzyme is a DNAzyme or a ribozyme.

In some embodiments, the antisense sequence of a nucleic acid molecule is an antisense sequence of a viral nucleic acid sequence or an antisense sequence of a bacterial nucleic acid sequence. In some embodiments, the antisense sequence of a nucleic acid molecule is an antisense sequence of a viral nucleic acid sequence. In some embodiments, the antisense sequence of a viral sequence is

[SEQ ID NO: 10]
AACGTCGGATCCCGCGTCGCC/3InvdT/.

In some embodiments, the viral nucleic acid sequence is a hepatitis C viral sequence. In some embodiments, the viral nucleic acid sequence is GGCGACGCGGGATCCGACGTT [SEQ ID NO:11]. In some embodiments, the viral nucleic acid sequence is GCCGATGGGGGATGTTCCGGA [SEQ ID NO:12]. In some embodiments, the viral nucleic acid sequence is GTTGACGCGCAAACCTACGTC [SEQ ID NO:13].

In some embodiments, the nucleic acid aptamer, the nucleic acid enzyme and the antisense sequence of a nucleic acid molecule interacts with and binds their respective analytes through structural recognition. Upon binding of the analyte, the nucleic acid aptamer, the nucleic acid enzyme and the antisense sequence of a nucleic acid molecule undergo a conformational change which trigger the release of the nucleic acid aptamer, the nucleic acid enzyme or the antisense sequence of a nucleic acid molecule from the nucleic acid assembly.

In some embodiments, the analyte is selected from, but not limited to, small inorganic molecules, small organic molecules, metal ions, hormonal growth factors, biomolecules, toxins, biopolymers (such as carbohydrates, lipids, peptides and proteins), cells, tissues and microorganisms (including bacteria and viruses). In an embodiment, the analyte is either isolated from a natural source or is synthetic. The term analyte also includes mixtures of compounds or agents such as, but not limited to, combinatorial libraries and samples from an organism or a natural environment. In some embodiments, the biosensor of the present application is used to detect analytes that are small molecules, proteins or DNA.

In some embodiments, the analyte which binds a DNA aptamer is a nucleotide triphosphate (NTP). In some embodiments, the nucleotide triphosphate is selected from adenosine triphosphate (ATP), guanosine triphosphate (GTP), cytidine triphosphate (CTP), 5-methyluridine triphosphate (m⁵UTP), uridine triphosphate (UTP) and adenosine monophosphate (AMP). In some embodiments, the NTP is selected from deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP).

In some embodiments, the first nucleic acid sequence and the second nucleic acid sequence of the linear single-stranded nucleic acid sequence is a pre-primer sequence. In some embodiments, the pre-primer sequence is

[SEQ ID NO: 3]
GTTAC AGTCA CGGTA TATTT ACCCA GGTCA GTG.

In some embodiments, the pre-primer sequence is

[SEQ ID NO: 4]
GTTAC AGTCA CGGTA TATTTAGCCG TAGCC TG.

In some embodiments, the pre-primer sequence is

[SEQ ID NO: 5]
GTTAC AGTCA CGGTA TATTTAGGATCCGACGTT.

In some embodiments, the pre-primer sequence is GTTAC AGTCACGGTA TATTT ACCCA GGTCA GTG/3invdT/[SEQ ID NO:6].

In some embodiments, the RCA is an isothermal enzymatic process where short DNA or RNA primers are amplified to form a long single-stranded DNA or RNA using a circular DNA template and an appropriate DNA or RNA polymerase. In some embodiments, the RCA is HRCA, which is a technique derived from rolling circle amplification to improve upon the sensitivity of RCA by using both forward and reverse primers. The forward primer produces a multimeric single-stranded DNA (ssDNA) or single-stranded RNA (ssRNA), which then becomes the template for multiple reverse primers. The DNA or RNA polymerase then extends the reverse primer during the extension process and the downstream DNA or RNA is displaced to generate branching or a ramified DNA or RNA complex. When all ssDNA and ssRNA strands have been converted into double-stranded DNA (dsDNA) or double-stranded RNA (dsRNA), the process ceases

In some embodiments, the biosensor of the application further comprises a nucleic acid polymerase. In some embodiments, the nucleic acid polymerase is a DNA polymerase having 3′ to 5′ exonuclease activity or an RNA polymerase having 3′ to 5′ exonuclease activity. In some embodiments, the nucleic acid polymerase is a DNA polymerase. In some embodiments, the nucleic acid polymerase is φ29DP.

In some embodiments, the circular single-stranded nucleic acid molecule that is the RCA template forms a nucleic acid duplex with the first nucleic acid sequence of the linear single-stranded nucleic acid molecule which acts as a primer sequence for the RCA template.

In some embodiments, the second nucleic acid sequence that is digested by the nucleic acid polymerase having exonuclease activity of the linear single-stranded nucleic acid molecule forms a nucleic acid duplex with the linear single-stranded nucleic acid molecule that binds the analyte.

In some embodiments the range of detection of the biosensors of the application is less than nanomolar concentrations of the analyte. In some embodiments the range of detection of the biosensors of the application is less than picomolar concentrations of the analyte. In some embodiments the range of detection of the biosensors of the application is less than femptomolar concentrations of the analyte. In some embodiments the range of detection of the biosensors of the application is less than attomolar concentrations of the analyte. In some embodiments the range of detection of the biosensors of the application is between attomolar and nanomolar concentrations of the analyte.

### III. Methods of the Application

The present application also includes assay methods that utilize the biosensor of the present application. In an embodiment, the assay is a method of detecting an analyte in a sample, wherein the sample comprises or is suspected of comprising the analyte, the method comprising contacting the sample with the biosensor of the application and monitoring for a presence of a nucleic acid product from the RCA template wherein the presence of the nucleic acid product from the RCA template indicates the presence of the analyte in the sample.

The sample is from any source, for example, any biological (for example human or animal medical samples), environmental (for example water or soil) or natural (for example plants) source, or from any manufactured or synthetic source (for example food or drinks). It is most convenient for the sample to be a liquid or dissolved in a suitable solvent to make a solution. For quantitative assays, the amount of sample in the solution should be known. The sample is one that comprises or is suspected of comprising one or more analytes.

In an embodiment, the analyte is either isolated from a natural source or is synthetic. The term analyte also includes mixtures of compounds or agents such as, but not limited to, combinatorial libraries and samples from an organism or a natural environment. In some embodiments, the biosensor of the present application is used to detect analytes that are small molecules, proteins or DNA.

In some embodiments, the analyte is selected from small inorganic molecules, small organic molecules, metal ions, hormonal growth factors, biomolecules, toxins, biopolymers (such as carbohydrates, lipids, peptides and proteins), microorganisms (including bacteria and viruses), cells and tissues. In another embodiment, the analyte is selected from small inorganic molecules, small organic molecules, hormonal growth factors, biomolecules, peptides, proteins, bacteria, viruses and cells. In some embodiments, the analyte is selected from hormonal growth factors, viruses and biomolecules. In some embodiments, the analyte is a biomolecule. In some embodiments, the analyte is a hormonal growth factor. In some embodiments, the analytes is a nucleic acid sequence of a bacterial or a viral genome. In some embodiments, the analyte is adenosine triphosphate. In some embodiments, the analyte is platelet-derived growth factor. In some embodiments, the analyte is a DNA sequence of the hepatitis C viral genome.

In some embodiments, the nucleic acid product from the RCA template is a single-stranded DNA molecule or a single-stranded RNA molecule. In some embodiments, the nucleic acid product from the RCA template is a long single-stranded DNA molecule or a single-stranded RNA molecule comprising repeating nucleic acid sequences. In some embodiments, the term long refers to nucleic acid sequences comprising thousands of repeating sequence units.

In some embodiments, the nucleic acid product from the RCA template is generated through rolling circle amplification. The rolling circle amplification reaction is performed in the presence of the biosensor of the present application, an RCA reaction buffer, deoxynucleotides (dNTPs), a nucleic acid polymerase and a suitable solvent. The circular single-stranded nucleic acid molecule, the linear single-stranded nucleic acid molecule that binds the analyte, the linear single-stranded nucleic acid molecule comprising the first nucleic acid sequence and the second nucleic acid sequence, are incubated at a temperature and time sufficient for the formation of the nucleic acid assembly of the biosensor. Examples of non-limiting reaction temperatures include, but are not limited to, 10° C. to about 30° C. or about 20° C. to about 25° C. Examples of non-limiting reaction times include, but are not limited to, 5 minutes to about 1 hour or about 15 minutes to about 30 minutes.

Subsequently, the RCA reaction is initiated by the addition of the RCA reaction buffer, dNTPs, the nucleic acid polymerase and the suitable solvent. The reaction mixture is incubated at a first temperature and time and then subjected to a second temperature and time sufficient to complete the RCA process. Examples of non-limiting temperatures for the first temperature include, but are not limited to, 10° C. to about 40° C. or about 20° C. to about 30° C. Examples of non-limiting reaction times for the first time period include, but are not limited to, 30 minutes to about 3 hours or about 1 hour to about 2 hours. Examples of non-limiting temperatures for the second temperature include, but are not limited to, 50° C. to about 120° C. or about 70° C. to about 90° C. Examples of non-limiting reaction times for the second time period include, but are not limited to, 1 minute to about 30 minutes or about 5 minutes to about 20 minutes. In some embodiments, the suitable solvent is an aqueous solvent. In some embodiments, the aqueous solvent is water. In some embodiments, the nucleic acid polymerase is a DNA polymerase having exonuclease activity, in particular 3′-5′ exonuclease activity. In some embodiments, the DNA polymerase is ϕ29DP.

Each round of the RCA process generates a nucleic acid product. The nucleic acid product is a multimeric single-stranded nucleic acid product. In some embodiments, the multimeric single-stranded nucleic acid product further serves as a RCA template.

In some embodiments, the nucleic acid product from the RCA template is generated through hyper-branched rolling circle amplification (HRCA). The HRCA reaction is performed in the presence of the biosensor of the present application, RCA reaction buffer, deoxynucleotides (dNTPs), a saturating intercalating fluorescent dye, reverse primer sequences, a nucleic acid polymerase and a suitable solvent. In some embodiments, the HRCA process is carried out in cuvettes placed in a fluorimeter set at a constant temperature wherein fluorescent intensity is measured at time intervals sufficient for a fluorescence maxima plateau to be reached. Examples of non-limiting reaction temperatures include, but are not limited to, 10° C. to about 50° C. or about 20° C. to about 30° C. In some embodiments, the HRCA reaction is monitored in 1 minute time intervals. In some embodiments, the suitable solvent is an aqueous solvent. In some embodiments, the aqueous solvent is water. In some embodiments, the nucleic acid polymerase is a DNA polymerase having exonuclease activity, in particular 3′-5′ exonuclease activity. In some embodiments, the DNA polymerase is ϕ29DP.

In some embodiments, the detection of the analyte is performed by monitoring for the presence of a nucleic acid product. In this embodiment, the nucleic acid product being formed possesses a detectable signal (for e.g., fluorescence, molecular weight) that is distinct from the signal of any of the starting reagents.

In some embodiments, the presence of the nucleic acid product comprises a detection system. In an embodiment, the detection system is selected from a fluorescent system, a colorimetric system, an electrophoresis system and an electrochemical system.

In some embodiments, the presence of the nucleic acid product from the RCA template is monitored using an electrophoresis system and the presence of the analyte is confirmed by detection of a single molecular weight band. The process of preparing the sample, preparing the gel and subsequent visualization techniques of the electrophoresis system are well known in the prior art.

In some embodiments, the nucleic acid products from the RCA template are measured using nucleic acid electrophoresis. In some embodiments, the nucleic acid electrophoresis is conducted under denaturing conditions. In some embodiments, the electrophoresis system is selected from denaturing polyacrylamide gel electrophoresis (dPAGE) and agarose gel electrophoresis.

In some embodiments, the presence of the nucleic acid product from the RCA template is monitored using a fluorescent system and the presence of the analyte is confirmed by detection of a fluorescent signal.

In some embodiments, the fluorescent system comprises a fluorescent reporter molecule that monitors the progression of the nucleic acid product amplification. Depending on the mode of signal generation, the fluorescent reporter molecule is either a fluorogenically labelled oligonucleotide, referred to as a probe, or a fluorogenic nucleotide-binding dye.

In an embodiment, the selection of the fluorescent reporter molecule for the biosensor is based upon one or more parameters including, but not limited to, (i) maximum excitation and emission wavelength, (ii) extinction coefficient, (iii) quantum yield, (iv) lifetime, (v), stokes shift, (vi) polarity of the fluorophore and (vii) size.

In some embodiments, the fluorescent reporter molecule is a high resolution melting (HRM) dye or probe. The HRM analysis provides the capabilities of monitoring the presence of nucleic acid production in real-time. The HRM dyes are saturating intercalating fluorescent dyes which upon binding in high amounts to double-stranded nucleic acids produce a bright fluorescent signal. In some embodiments, the fluorescent system comprises a saturating nucleic acid intercalating fluorescent dye. In some embodiments, the saturating nucleic acid intercalating fluorescent dye is a cyanine dye, for example selected from LC Green™, P2, SYTO9™, Eva Green™ Chromofy™, BEBO™, SYBR Gold™ and BOXTO™. In some embodiments, the saturating nucleic acid intercalating fluorescent dye is Eva Green™.

In some embodiments, when the sample comprises the analyte, contacting the sample with the biosensor of the present application induces:
(a) binding of the analyte to the analyte-binding single-stranded nucleic acid molecule causing the release of the analyte-binding single-stranded nucleic acid sequence from the second nucleic acid sequence of the linear single-stranded nucleic acid molecule;
(b) an exonucleolytic digestion of the second nucleic acid sequence by the nucleic acid polymerase resulting in a mature primer nucleic sequence comprising the first nucleic acid sequence; and
(c) binding of the nucleic acid polymerase to the mature primer nucleic acid sequence to initiate rolling circle amplification (RCA) using the circular single-stranded nucleic acid molecule to produce the single-stranded nucleic acid product being monitored.

In some embodiments the range of detection of the biosensors of the application is less than nanomolar concentrations of the analyte. In some embodiments the range of detection of the biosensors of the application is less than picomolar concentrations of the analyte. In some embodiments the range of detection of the biosensors of the application is less than femptomolar concentrations of the analyte. In some embodiments the range of detection of the biosensors of the application is less than attomolar concentrations of the analyte. In some embodiments the range of detection of the biosensors of the application is between attomolar and nanomolar concentrations of the analyte.

The present application further includes kits comprising the biosensors of the application. In some embodiments, the kit includes the biosensor and any further reagents for performing an assay using the biosensor, for example a nucleic acid polymerase having exonuclease activity. In some embodiments, reagents include a RCA reaction buffer, deoxynucleotides (dNTPs), a saturating nucleic acid intercalating fluorescent dye and water. In some embodiments, the dNTPs are dATP, dGTP, dCTP, dTTP and dUTP.

In some embodiments, the kit includes instructions for using the biosensor in the assay and any controls needed to perform the assay. The controls may be on the biosensor itself, or alternatively, on a separate substrate. In some embodiments, control reactions lack the circular single-stranded nucleic acid molecule, the linear single-stranded nucleic acid molecule that binds the analyte, the linear single-stranded nucleic acid molecule comprising the first nucleic acid sequence or the second nucleic acid sequence, or combinations thereof.

In some embodiments, the kit includes all the components required to perform any of the assay methods of the present application.

### EXAMPLES

The following non-limiting examples are illustrative of the present application:

### Example 1: Development of Biosensors Comprising a Nucleic Acid Assembly

Oligonucleotides and Other Materials

The sequences of all DNA molecules are provided in Table 1. DNA oligonucleotides were obtained from Integrated DNA Technologies (IDT, Coralville, Iowa, USA), and purified by 10% denaturing (8 M urea) polyacrylamide gel electrophoresis (dPAGE). T4 polynucleotide kinase (PNK), T4 DNA ligase, ϕ29DP, ATP and dNTPs were purchased from Thermo Scientific (Ottawa, ON, Canada). α-[³²P]ATP was acquired from PerkinElmer (Woodbridge, ON, Canada). Water was purified with a Milli-Q Synthesis A10 water purification system. All other chemicals were purchased from Sigma-Aldrich (Oakville, Canada) and used without further purification.

Instruments

The autoradiograms and fluorescent images of dPAGE and agarose gels were obtained using a Typhoon 9200 variable mode imager (GE Healthcare) and analyzed using Image Quant software (Molecular Dynamics). Fluorescence measurements were performed using a Cary Eclipse fluorescence spectrophotometer (Varian) with an excitation wavelength of 500 nm and emission wavelength of 530 nm.

Preparation of Circular Template CT1

Circular template (CT) was prepared from a 5′-phosphorylated linear template LT1 through circularization with T4 DNA ligase and circularization DNA template CDT1. To phosphorylate LT1 at the 5′-end, 200 pmol of LT1 was mixed with 10 U (U: unit) of PNK and 2 mM ATP in 50 μL of 1×PNK buffer A (50 mM Tris-HCl, pH 7.6 at 25° C., 10 mM MgCl₂, 5 mM DTT, 0.1 mM spermidine), incubated at 37° C. for 40 min, and heated at 90° C. for 5 min. To circularize 5′-phosphorylated LT1, 300 pmol of CDT1 was added into the reaction mixture above. This mixture was heated at 90° C. for 5 min and cooled at room temperature (˜23° C.) for 20 min. This was followed by the addition of 15 μL of 10×T4 DNA ligase buffer (400 mM Tris-HCl, 100 mM MgCl₂, 100 mM DTT, 5 mM ATP, pH 7.8 at 25° C.) and 10 U of T4 DNA ligase. The resultant mixture (150 μL in total) was incubated at room temperature for 2 h and then heated at 90° C. for 5 min to deactivate the ligase. The DNA in the mixture was concentrated by ethanol precipitation and the CT1 in the mixture was purified by 10% dPAGE.

Exonucleolvtic Digestion of PP1, PP2 and PP3

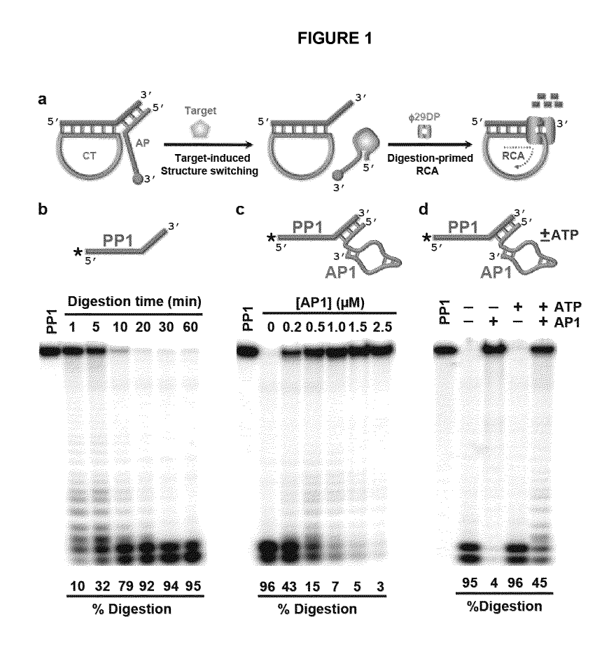

For digestion of pre-primer (PP) 1 alone (FIG. 1B), 1 μM 5′-³²P labeled PP1 was incubated at 30° C. with 1 μL of the ϕ29DP stock (5 U/μL) in 50 μL of 1×RCA reaction buffer (33 mM Tris acetate, 10 mM magnesium acetate, 66 mM potassium acetate, 0.1% (v/v) Tween-20, 1 mM DTT, pH 7.9 at 25° C.). 5 μL of the reaction mixture was taken at 1, 5, 10, 20, 30 and 60 min, combined with 5 μL of urea-based 2× denaturing gel loading buffer, heated at 90° C. for 5 min, and analyzed by 20% dPAGE.

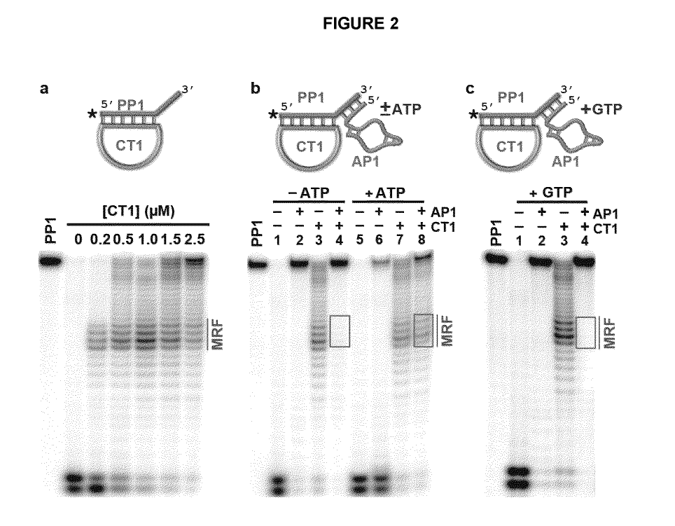

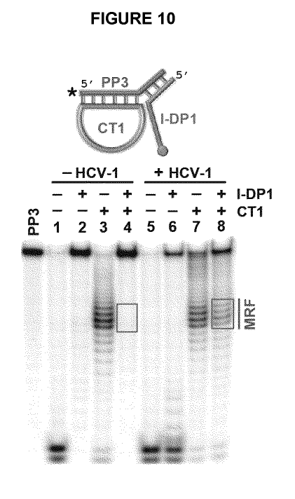

For pre-primer 1 (PP1)-aptamer 1 (AP1) hybrid digestion (FIG. 10), the DNA hybridization was performed in 40 μL of hybridization buffer (50 mM Tris-HCl, pH 7.4 at 25° C., 100 mM NaCl, 5 mM MgCl₂ and 0.02% Tween-20) containing 50 pmol of 5′-³²P labeled PP1 and varying amounts of 5′-FAM labeled AP1. The mixture was heated at 90° C. for 5 min and cooled to room temperature for 20 min. To initiate the digestion, 1 μL of the ϕ29DP stock, 5 μL of 10×RCA reaction buffer and 4 μL of water were added. The final concentration of PP1 was 1 μM and that of AP1 was 0.2, 0.5, 1.0, 1.5 or 2.5 μM. The reaction mixtures were incubated at 30° C. for 30 min before the addition of an equal volume of 2× denaturing gel loading buffer and heating at 90° C. for 5 min. The resultant mixtures were analyzed by 20% dPAGE. The PP1-CT1 hybrid was digested and analyzed in the same way other than the replacement of AP1 with CT1 (FIG. 2A).

For the digestion of PP1-AP1 and PP1-AP1-CT1 in the presence of ATP or GTP (FIG. 1D, 2B, 2C, 7), the hybridization reaction was performed in 40 μL of hybridization buffer containing 50 pmol of 5′-³²P labeled PP1 and 75 pmol of AP1 (for PP1-AP1) as well as 50 pmol of CT1 (for PP1-AP1-CT1) using the same procedure described above. Then 5 μL of 10×RCA reaction buffer, 1 μL of 25 mM ATP or GTP, 3 μL of water and 1 μL of the ϕ29DP stock were added. Various control reactions lacking AP1, CT1, ATP, GTP or a combination of these were also set up in the same way. Each reaction mixture was incubated at 30° C. for 30 min and then subjected to 20% dPAGE analysis using the identical procedure as described above.

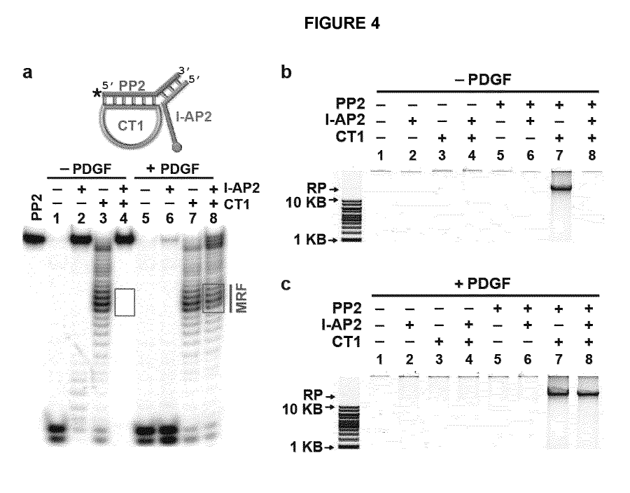

For digestion of PP2 alone or in the I-AP2-PP2 and I-AP2-PP2-CT1 hybrids with and without PDGF (FIG. 4A), the procedure was similar to the one used for the ATP system. The reaction mixture contained various combinations of 1 μM radioactive PP2, 1 μM CT1, 1.5 μM I-AP2, 100 nM PDGF and 0.1 U/μL ϕ29DP. Each reaction mixture was incubated at 30° C. for 30 min and analyzed by 20% dPAGE.

The digestion of PP3 alone or in the hybrid of I-DP1-PP3 and I-DP1-PP3-CT1 with and without HCV-1 DNA (FIG. 10) was also carried out similarly. The reaction mixture contained various combinations of 1 μM radioactive PP3, 1 μM CT1, 1.5 μM I-DP1, 100 nM I-HCV-1 DNA and 0.1 U/μL ϕ29DP. Each reaction mixture was incubated at 30° C. for 30 min and analyzed by 20% dPAGE.

RCA Reaction

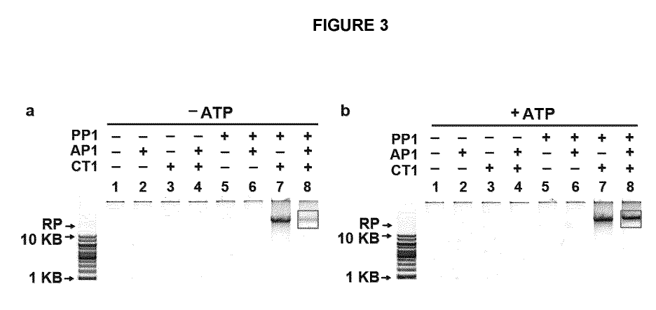

For the RCA reaction with the ATP sensing system (FIG. 3), following the hybridization reaction with 50 pmol of PP1, 50 pmol of CT1 and 75 pmol of AP1 in 40 μL of hybridization buffer, 1 μL of 25 mM ATP was added, and the resultant mixture was incubated at room temperature for 30 min. The RCA reaction was then initiated by the addition of 5 μL of 10×RCA reaction buffer, 2 μL of dNTPs (10 mM each of dATP, dCTP, dGTP and dTTP), 1 μL of ϕ29DP stock, and 2 μL of water. The reaction mixtures were incubated at 30° C. for 1 h before heating at 90° C. for 5 min. Various control reactions lacking PP1, CT1, ATP, or a combination of these were also set up in the same way. The RCA products from these reactions were analyzed by 0.6% agarose gel electrophoresis.

For the RCA reaction with the PDGF sensing system (FIGS. 4B and C), the procedure was identical to that used for the ATP-induced reaction except for the substitution of I-AP2 for AP1, PP2 for PP1, and PDGF (100 nM as the final concentration) for ATP. BSA, thrombin and IgG were also used as non-target controls. The concentration of PDGF was tested at 0.001, 0.005, 0.01, 0.05, 0.1, 0.5, 1, 5, 10 and 50 nM.

Detection of PDGF and HCV-1 DNA Using HRCA Reactions

Following the hybridization between 50 pmol of PP2, 50 pmol of CT1 and 75 pmol of I-AP2 in 30 μL of hybridization buffer, 5 μL of 10×RCA reaction buffer, 1 μL of a given PDGF stock solution, 1 μL of ϕ29DP stock, 2 μL of dNTPs (10 mM each), 2 μL of FP1 (10 μM), 2 μL of RP1 (10 μM) and 2.5 μL of 25× EvaGreen and 4.5 μL of water were added. These reactions were carried out in cuvettes placed in the fluorimeter set to a constant temperature of 30° C., and the fluorescence intensity was recorded in 1 min intervals.

The procedure for the HCV-1 DNA detection was similar to that for the PDGF detection except that the reagents were used as follows: 50 pmol of PP3 and 75 pmol of I-DP1, with HCV-1 concentration varying between 2 aM-20 nM.

Results and Discussion

Aptamer AP1

Using the well-known anti-ATP DNA aptamer,^{[13]} the digestion of (pre-primer) PP in the absence and presence of aptamer (AP) was assessed. The AP and PP for ATP detection are named AP1 and PP1, respectively (sequences of the DNA molecules used for this work are provided in Table 1). As shown in FIG. 1B, more than 90% of PP1 (1 μM) was degraded by ϕ29DP (0.1 units/μL) within 30 minutes. However, degradation of PP1 was decreased to 3% in the presence of 2.5 μM AP1 (FIG. 10). The results indicate that AP1 can indeed block nucleolytic digestion of PP1 by ϕ29DP through AP1-PP1 duplex formation.

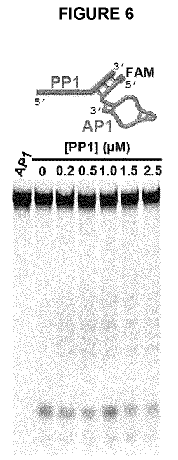

AP1 is rather resistant to nucleolytic digestion by ϕ29DP as <5% was digested after 60 minutes (FIG. 6), compared to 96% for PP1 under the same conditions (FIG. 1A). This indicates that the aptamer has a structure that is resistant to exonucleolytic digestion by ϕ29DP, consistent with the reported hairpin structural model of the aptamer.^{[13b]}

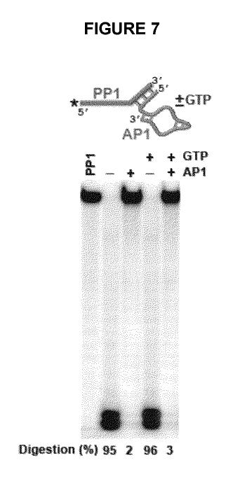

The effect of ATP on PP1 digestion was subsequently assessed, expecting that ATP would induce AP1 release from the AP1-PP1 duplex by structure switching.^{[12]} Indeed, addition of ATP (0.5 mM) led to significantly increased cleavage of PP1 in the presence of AP1 (45%, vs. 4% without ATP; FIG. 1D). In contrast, no significant change was observed in PP1 digestion when GTP was supplied (FIG. 7). This result shows that AP1 release is ATP-dependent.

The digestion of PP1 (1 μM) in the presence of CT1 (0-2.5 μM, FIG. 2A) was next investigated. The PP1 digestion pattern was changed when CT1 was supplied: with increasing concentrations of CT1, the amount of small digestion products was reduced whereas the quantity of mid-range fragments (denoted MRF) was increased (FIG. 2A). This observation is consistent with the expectation that the unpaired region of PP1 was trimmed by ϕ29DP.

The digestion pattern of PP1 within the PP1-AP1-CT1 assembly was then studied. In the absence of ATP, PP1 was protected from exonucleolytic digestion by ϕ29DP, as no MRF were observed (FIG. 2B, lane 4, box). However, addition of ATP resulted in trimming of the exposed 3′-end, reflected by the appearance of MRF (FIG. 2B, lane 8, box). When ATP was replaced with GTP, MRF disappeared (FIG. 2C, lane 4, box).

FIGS. 1 and 2 illustrate that (1) ϕ29DP can digest ss PP1, (2) formation of the PP1-AP1 duplex blocks PP1 digestion; (3) addition of ATP promotes release of AP1 from the tripartite assembly; and (4) ϕ29DP trims the exposed ss fragment of PP1, converting it into the mature primer.

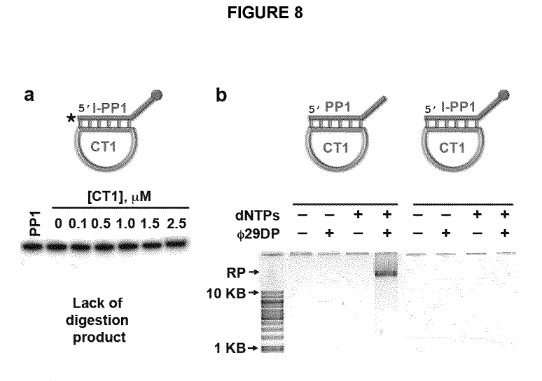

To illustrate that nucleolytic trimming of PP1 can result in a mature primer that can initiate RCA, the RCA reaction was carried out with the PP1-CT1 hybrid. As a control, the same reaction with I-PP1-CT1, a modified PP1 containing an inverted dT at the 3′-end, was performed. This modification should render I-PP1 completely resistant to digestion by ϕ29DP. Indeed, it was found that ϕ29DP was incapable of degrading I-PP1 (FIG. 8A). Agarose gel analysis indicated that RCA product (RP) was produced when PP1 was incubated with CT1, dNTPs and ϕ29DP (FIG. 8B). However, RP was not observed when I-PP1 was used to replace PP1. These results show that successful trimming of PP1 by ϕ29DP is a prerequisite for RCA.

The ATP-promoted RCA reaction of the PP1-AP1-CT1 assembly was next assessed. Three events were expected to occur: (1) ATP-promoted structure switching, (2) exonucleolytic trimming of PP1 by ϕ29DP, and (3) RCA by ϕ29DP. The structure-switching event (mixing the DNA assembly with ATP) was separated from the primer trimming and RCA events (mixing ATP/DNA solution with ϕ29DP/dNTPs). The results are shown in FIG. 3 (panel a: −ATP; panel b: +ATP). The first 6 lanes of each panel serve as negative controls (RCA should not occur when PP1 or CT1 is omitted). Each lane 7 serves as a positive control (RCA should occur when both PP1 and CT1 are provided but AP1 is omitted). The final lane of each panel serves as the ATP-dependence test. As expected, no RP was observed in any of the negative controls but was found in the two positive controls. More importantly, the presence of ATP indeed resulted in significantly more RP production: the RP band in lane 8 of panel b is much more intense than the same band in panel a (indicated by the boxes). Without wishing to be bound by theory, RP should not have been observed in the absence of ATP. However, it is known that the DNA aptamer can also bind dATP.^{[13]} Therefore, the small amount of RP in the absence of ATP is likely to have originated from the nucleolytic trimming-RCA step where dATP was supplied as part of the dNTPs needed for DNA amplification. This is also the reason that the structure-switching step was separated from the trimming and RCA steps.

Aptamer AP2

To demonstrate that ligand-responsive RCA was a general feature for structure-switching aptamers, another aptamer system was investigated. A new DNA aptamer probe, AP2, based on a reported aptamer that binds human platelet-derived growth factor (PDGF) was investigated.^{[14]} To prevent the degradation by ϕ29DP, AP2 was modified with an inverted dT at the 3′-end (named I-AP2) as this aptamer does not have an intrinsic structure resistant to nucleolytic digestion of ϕ29DP.

The tripartite assembly is made of I-AP2-PP2-CT1. Digestion of radioactive PP2 was carried out under various conditions and the results were nearly identical to the ATP system (FIG. 4A). Briefly, in the absence of I-AP2 and CT1, PP2 was fully digested (lanes 1 and 5). When I-AP2 was provided but CT1 was omitted, PP2 was very much protected in the absence of PDGF (lane 2) but largely digested in the presence of PDGF (lane 6). However, when CT1 was provided but I-AP2 was omitted, PP2 was partially digested into MRF both in the absence (lane 3) and presence (lane 7) of PDGF. More importantly, when both I-AP2 and CT1 were provided, PP2 was fully protected in the absence of PDGF (lane 4), but trimmed into MRF in the presence of PDGF (lane 8, box).

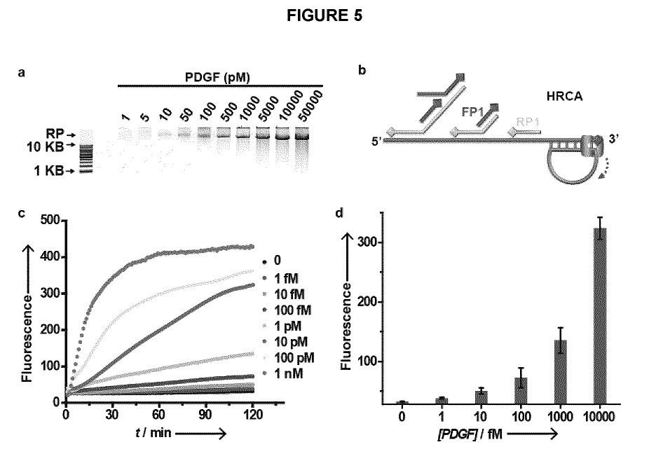

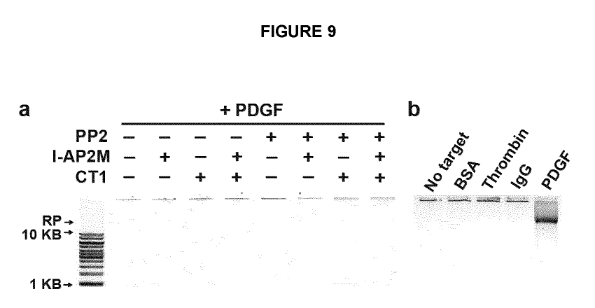

The results of the RCA reaction of the I-AP2-PP2-CT1 assembly are shown in FIGS. 4B and C. In contrast to the ATP system, structure switching, nucleolytic trimming and RCA reactions can be performed simultaneously. As expected, in the absence of PDGF, the RCA reaction was arrested (FIG. 4B, lane 8; lanes 1-7 serve as various controls, as in the case of the ATP system). However, RP was observed upon addition of PDGF (FIG. 4C; lane 8). Control experiments with other proteins (BSA, thrombin and IgG) and a mutant DNA aptamer (I-AP2M) demonstrated that the RCA reaction was dependent both on the matching target for the aptamer (FIG. 9A) and the specific aptamer sequence (FIG. 9B). These results demonstrate that stimuli-responsive, digestion-primed RCA can be generally adopted for structure-switching aptamers. Further, the production of RP in response to increasing concentrations of PDGF was analyzed. As shown in FIG. 5A, as low as 10 pM can be detected by agarose gel analysis.

To further improve detection sensitivity, hyper-branched RCA (HRCA) was employed.^{[15]} In HRCA (as illustrated in FIG. 5B), DNA products generated from RCA using a forward primer (FP1) are further copied by ϕ29DP using a second primer (reverse primer, RP1) into DNA products that can be further amplified using FP1. This process results in an exponential amplification.^{[16]} This strategy was adopted with the use of FP1 and RP1 as the cross-amplification primers. The DNA intercalating dye Eva Green™ was used to achieve real-time monitoring of HRCA products. In the presence of PDGF, fluorescence intensity increased gradually with reaction time, indicating that PDGF can indeed initiate HRCA (FIG. 5C). Using this method, PDGF can be detected at a concentration as low as 1 fM (FIG. 5D). Remarkably, HRCA offers a detection sensitivity that is 4 orders of magnitude better than that of regular RCA (10 pM). The PDGF aptamer has a dissociation constant (K_{d}) of ˜0.1 nM^{[14]} and the previously reported structure-switching fluorescent aptamer biosensor was only able to achieve a detection limit of ˜2 nM.^{[17a]} Therefore, the biosensing strategy as taught in the present application offers a dramatically improved detection limit. To the best of the Applicant's knowledge, the 1 fM limit of detection represents the lowest detected concentration ever achieved with the PDGF aptamer.^{[7b,17]}

DNA Probe DP1

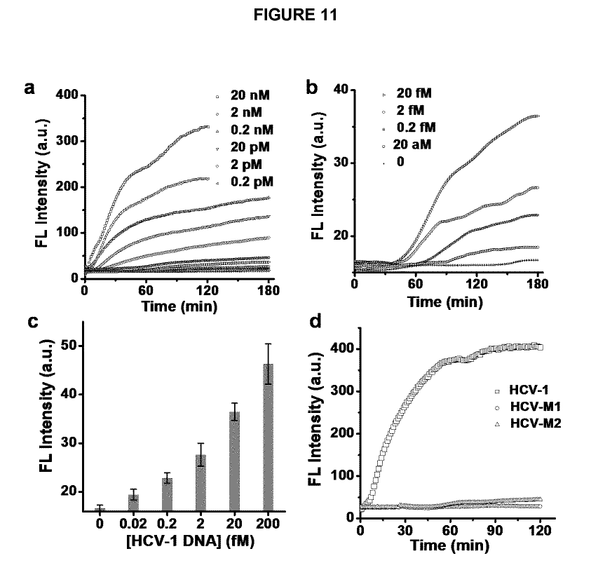

To extend the digestion-primed RCA approach beyond aptamer-based detection, the same strategy was applied for DNA detection (FIGS. 10 and 11). The DNA probe, I-DP1, has a specific DNA sequence designed to recognize HCV-1 DNA, representing a portion of the complementary DNA sequence from the hepatitis C virus genome.^{[18]} Once again, the HRCA strategy was adopted, along with the use of Eva Green™ for real-time detection of DNA amplicons. The fluorescence intensity increased in response to HCV-1 DNA in a time-dependent manner (FIGS. 11A and B). The limit of detection, established by plotting fluorescence intensity obtained at 180 minutes vs. DNA concentration (FIG. 11C), was found to be 20 aM, corresponding to 600 copies of DNA in 50 μL. Besides the outstanding detection limit, this method also exhibited excellent selectivity. No increase of fluorescence was observed when the system was tested with unintended DNA targets, such as HCV-M1 and HCV-M2 (containing 7 and 9 mismatched nucleotides, respectively; FIG. 11D).

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 3

| Name of DNA oligonucleotide | Sequence (5′-3′) |
|---|---|
| Precursor of circular template 1 (CT1) | |
| LT1 (60 nt) | ACTGTAACCA TTCTT GTTTC GTATC ATTGC AGAATTCTAC TAATT |
| | TATCT GAATACCGTG |
| | [SEQ ID NO: 1] |
| Circularization DNA template | |
| CD1 (16 nt) | GTTAC AGTCA CGGTA T |
| | [SEQ ID NO: 2] |
| Pre-primers | |
| PP1 (Pre-primer 1, for ATP detection, 33 nt) | GTTAC AGTCA CGGTTA TATTT ACCCA GGTCA GTG |
| | [SEQ ID NO: 3] |
| PP2 (Pre-primer 2, for PDGF detection, 32 nt) | GTTAC AGTCA CGGTA TATTTAGCCG TAGCC TG |
| | [SEQ ID NO: 4] |
| PP3 (Pre-primer 3, for DNA detection, 33 nt) | GTTAC AGTCA CGGTA TATTTAGGATCCGACGTT |
| | [SEQ ID NO: 5] |
| I-PP1 (PP1 with an inverted dT at 3′-end) | GTTAC AGTCACGGTA TATTT ACCCA GGTCA GTG/3invdT/ |
| | [SEQ ID NO: 6] |
| ATP probe | |
| AP1 (32 nt) | CACTG ACCTG GGGGA GTATT GCGGA GGAAGGT |
| | [SEQ ID NO: 7] |
| PDGF probe | |
| I-AP2 (35 nt | CAGGC TACGG CACGT AGAGC ATCAC CATGA TCCTG/3invdT/ |
| | [SEQ ID NO: 8] |
| I-AP2M (35 nt, AP2 with mutations) | CAGGC TACGG CACTT TTTTC ATTTAAATTA TAATT/3invdT/ |
| | [SEQ ID NO: 9] |
| DNA probe | |
| I-DP1 (21 nt) | AACGTCGGATCCCGCGTCGCC/3InvdT/ |
| | [SEQ ID NO: 10] |
| DNA target | |
| HCV-1DNA (21 nt, target for DP1 | GGCGACGCGGGATCCGACGTT |
| | [SEQ ID NO: 11] |
| HCV-M1DNA (21 nt, HCV-1 with mutations | GCCGATGGGGGATGTTCCGGA |
| | [SEQ ID NO: 12] |
| HCV-M2 DNA (21 nt, HCV-1 with mutations) | GTTGACGCGCAAACCTACGTC |
| | [SEQ ID NO: 13] |
| Primers for HRCA | |
| FP1 (Forward primer 1, 16 nt) | GTTAC AGTCA CGGTA T |
| | [SEQ ID NO: 14] |
| RP1 (Reverse primer 1, 18 nt) | CATTGCAGAATTCTACTA |
| | [SEQ ID NO: 15] |

### REFERENCES

[1] R. K. Saiki, S. Scharf, F. Faloona, K. B. Mullis, G. T. Horn, H. A. Erlich, N. Arnheim, Science 1985, 230, 1350-1354.
[2] a) W. Zhao, M. M. Ali, M. A. Brook, Y. Li, Angew. Chem. Int. Ed. 2008, 47, 6330-6337; Angew. Chem. 2008, 120, 6428-6436; b) M. M. Ali, F. Li, Z. Zhang, K. Zhang, D. K. Kang, J. A. Ankrum, X. C. Le, W. Zhao, Chem. Soc. Rev. 2014, 43, 3324-3341; c) F. Wang, C. Lu, I. Willner, Chem. Rev. 2014, 114, 2881-2941.
[3] a) A. Fire, S. Q. Xu, Proc. Natl. Acad. Sci. USA 1995, 92, 4641-4645; b) D. Liu, S. L. Daubendiek, M. A. Zillman, K. Ryan, E. T. Kool, J. Am. Chem. Soc. 1996, 118, 1587-1594; c) X. Zhong, P. M. Lizardi, X. Huang, P. L. Bray-Ward, D. C. Ward, Proc. Natl. Acad. Sci. USA 2001, 98, 3940-3945.
[4] a) L. Blanco, A. Bernad, J. M. Lazaro, G. Martin, C. Garmendia, M. Salas, J. Biol. Chem. 1989, 264, 8935-8940; b) S. Kamtekar, A. J. Berman, J. Wang, J. M. Lazaro, M. de Vega, L. Blanco, M. Salas, T. A. Steitz, Mol. Cell. 2004, 16, 609-618.
[5] a) Y. Weizmann, M. K. Beissenhirtz, Z. Cheglakov, R. Nowarski, M. Kotler, I. Willner, Angew. Chem. Int. Ed. 2006, 45, 7384-7388; Angew. Chem. 2006, 118, 7544-7548; b) Y. Liu, H. Yao, J. Zhu, J. Am. Chem. Soc. 2013, 135, 16268-16271; c) Z. Wu, Z. Shen, K. Tram, Y. Li, Nat. Commun. 2014, 5, 4279; d) F. Wang, C. Lu, X. Liu, L. Freage, I. Willner, Anal. Chem. 2014, 86, 1614-1621.
[6] a) C. Larsson, I. Grundberg, O. Soderberg, M. Nilsson, Nat. Methods. 2010, 7, 395-397; b) Y. Wen, Y. Xu, X. Mao, Y. Wei, H. Song, N. Chen, Q. Huang, C. Fan, D. Li, Anal. Chem. 2012, 84, 7664-7669; c) R. Deng, L. Tang, Q. Tian, Y. Wang, L. Lin, J. Li, Angew. Chem. Int. Ed. 2014, 53, 2389-2393; Angew. Chem. 2014, 126, 2421-2425.
[7] a) D. A. Di Giusto, W. A. Wlassoff, J. J. Gooding, B. A. Messerle, G. C. King, Nucleic Acids Res. 2005, 33, e64; b) L. Yang, C. W. Fung, E. J. Cho, A. D. Ellington, Anal. Chem. 2007, 79, 3320-3329; c) L. Zhou, L. Ou, X. Chu, G. Shen, R. Yu, Anal. Chem. 2007, 79, 7492-7500; d) L. Wang, K. Tram, M. M. Ali, B. J. Salena, J. Li, Y. Li, Chem. Eur. J. 2014, 20, 2420-2424; e) Y. Mao, M. Liu, K. Tram, J. Gu, B. J. Salena, Y. Jiang, Y. Li, Chem. Eur. J. 2015, 21, 8069-8074; f) C. Carrasquilla, J. R. Little, Y. Li, J. D. Brennan, Chem. Eur. J. 2015, 21, 7369-7373.
[8] a) E. J. Cho, L. Yang, M. Levy, A. D. Ellington, J. Am. Chem. Soc. 2005, 127, 2022-2023; b) M. M. Ali, Y. Li, Angew. Chem. Int. Ed. 2009, 48, 3512-3515; Angew. Chem. 2009, 121, 3564-3567; c) S. A. McManus, Y. Li, J. Am. Chem. Soc. 2013, 135, 7181-7186.
[9] a) C. Lin, M. Xie, J. Chen, Y. Liu, H. Yan, Angew. Chem. Int. Ed. 2006, 45, 7537-7539; Angew. Chem. 2006, 118, 7699-7701; b) Z. Cheglakov, Y. Weizmann, A. B. Braunschweig, O. I. Wilner, I. Willner, Angew. Chem. Int. Ed. 2007, 47, 126-130; Angew. Chem. 2007, 120, 132-136; c) J. Lee, S. Peng, D. Yang, Y. H. Roh, H. Funabashi, N. Park, E. J. Rice, L. Chen, R. Long, M. Wu, D. Luo, Nat. Nanotechnol. 2012, 7, 816-820; d) G. Zhu, R. Hu, Z. Zhao, Z. Chen, X. Zhang, W. Tan, J. Am. Chem. Soc. 2013, 135, 16438-16445; e) Y. Ma, H. Zheng, C. Wang, Q. Yan, J. Chao, C. Fan, S. Xiao, J. Am. Chem. Soc. 2013, 135, 2959-2962.
[10] a) D. Zhang, B. Liu, Expert Rev Mol Diagn. 2003, 3, 237-248; b) V. Khare, K. A. Eckert, Mutation Res. 2002, 510, 45-54.
[11] a) B. D. Harfe, S. Jinks-Robertson, Annu. Rev. Genet. 2000, 34, 359-399; b) I. V. Shevelev, U. Hubscher, Nat. Rev. Mol. Cell Biol. 2002, 3, 364-376.
[12] a) R. Nutiu, Y. Li, J. Am. Chem. Soc. 2003, 125, 4771-4778; b) R. Nutiu, Y. Li, Chem. Eur. J. 2004, 10, 1868-1876; c) R. Nutiu, Y. Li, Angew. Chem. Int. Ed. 2005, 44, 1061-1065; Angew. Chem. 2005, 117, 1085-1089; d) P. S. Lau, B. K. Coombes, Y. Li, Angew. Chem. Int. Ed. 2010, 49, 7938-7942; Angew. Chem. 2010, 122, 8110-8114.
[13] a) D. E. Huizenga, J. W. Szostak, Biochemistry 1995, 34, 656-665; b) C. H. Li, D. J. Patel, Chem Biol. 1997, 4, 817-832.
[14] L. S. Green, D. Jellinek, R. Jenison, A. Ostman, C. H. Heldin, N. Janjic, Biochemistry 1996, 35, 14413-14424.
[15] a) D. Zhang, M. Brandwein, T. Hsuih, H. Li, Gene 1998, 211, 277-285; b) D. Zhang, M. Brandwein, T. Hsuih, H. Li, Mol. Diagn. 2001, 6, 141-150; c) Y. Cheng, X. Zhang, Z. Li, X. Jiao, Y. Wang, Y. Zhang, Angew. Chem. Int. Ed. 2009, 48, 3268-3272; Angew. Chem. 2009, 121, 3318-3322; d) A. Cao, C. Zhang, Anal. Chem. 2012, 84, 6199-6205.
[16] a) P. M. Lizardi, X. Huang, Z. Zhu, P. Bray-Ward, D. C. Thomas, D. C. Ward, Nat. Genet. 1998, 19, 225-232; b) D. Zhang, W. Zhang, X. Li, Y. Konomi, Gene 2001, 274, 209-216; c) G. Nallur, C. H. Luo, L. H. Fang, S. Cooley, V. Dave, J. Lambert, K. Kukanskis, S. Kingsmore, R. Lasken, B. Schweitzer, Nucleic Acids Res. 2001, 29, e118.
[17] a) F. Li, H. Zhang, C. Lai, X. Li, X. C. Le, Angew. Chem. 2012, 124, 9451-9454; Angew. Chem. Int. Ed. 2012, 51, 9317-9320; b) Y. Zhang, Y. Huang, J. Jiang, G. Shen, R. Yu, J. Am. Chem. Soc. 2007, 129, 15448-15449; c) C. Huang, Y. Huang, Z. Cao, W. Tan, H. Chang, Anal. Chem. 2005, 77, 5735-5741.
[18] M. Liu, J. Song, S. Shuang, C. Dong, J. D. Brennan, Y. Li, ACS Nano 2014, 8, 5564-5573.

1. A biosensor for detecting an analyte comprising a nucleic acid assembly wherein the nucleic acid assembly comprises:
(a) a circular single-stranded nucleic acid molecule that is a rolling circle amplification (RCA) template;
(b) a linear single-stranded nucleic acid molecule that binds the analyte; and
(c) a linear single-stranded nucleic acid molecule comprising a first nucleic acid sequence that is a primer for the RCA template and a second nucleic acid sequence that is digested by a nucleic acid polymerase having exonuclease activity,
wherein the first nucleic acid sequence of the linear single-stranded nucleic acid molecule binds to a portion of the circular single-stranded nucleic acid molecule and the second nucleic acid sequence of the linear single-stranded nucleic acid molecule binds to a portion of the analyte-binding single-stranded nucleic acid molecule in the absence of the analyte and in the presence of the analyte the binding of the second nucleic acid sequence of the linear single-stranded nucleic acid molecule to the portion of the analyte-binding single-stranded nucleic acid molecule is disrupted making the second nucleic acid sequence available for digestion by the nucleic acid polymerase having exonuclease activity.

2. (canceled)

3. The biosensor of claim 1, wherein (a), (b) and (c) are DNA molecules.

4. The biosensor of claim 1, wherein (a), (b) and (c) are RNA molecules.

5. The biosensor of claim 1, wherein (a), (b) and (c) comprise a combination of DNA and RNA molecules.

6. The biosensor of claim 1, wherein the linear single-stranded nucleic acid molecule that binds the analyte is selected from a nucleic acid aptamer, a nucleic acid enzyme and an antisense sequence of a nucleic acid molecule.

7. The biosensor of claim 6, wherein the nucleic acid aptamer is a DNA aptamer or an RNA aptamer.

8. The biosensor of claim 6, wherein the nucleic acid enzyme is a DNAzyme or a ribozyme.

9. The biosensor of claim 6, wherein the antisense sequence of a nucleic acid molecule is an antisense sequence of a viral nucleic acid sequence or an antisense sequence of a bacterial nucleic acid sequence.

10. The biosensor of claim 1, further comprising a nucleic acid polymerase.

11. The biosensor of claim 1, wherein the nucleic acid polymerase is a DNA polymerase having 3′ to 5′ exonuclease activity or an RNA polymerase having 3′ to 5′ exonuclease activity.

12. (canceled)

13. The biosensor of claim 11, wherein the nucleic acid polymerase is ϕ29DP.

14. A method of detecting an analyte in a sample, wherein the sample is suspected of comprising the analyte, the method comprising contacting the sample with the biosensor of claim 1, and monitoring for a presence of a nucleic acid product from the RCA template wherein the presence of the nucleic acid product from the RCA template indicates the presence of the analyte in the sample.

15. (canceled)

16. The method of claim 14, wherein the nucleic acid product from the RCA template is a single-stranded DNA molecule or a single-stranded RNA molecule.

17. The method of claim 14, wherein the presence of the nucleic acid product from the RCA template is monitored using an electrophoresis system and the presence of the analyte is confirmed by detection of a single molecular weight band.

18. (canceled)

19. The method of claim 14, wherein the presence of the nucleic acid product from the RCA template is monitored using a fluorescent system and the presence of the analyte is confirmed by detection of a fluorescent signal.

20. The method of claim 19, wherein the fluorescent system comprises a saturating nucleic acid intercalating fluorescent dye.

21. (canceled)

22. The method of claim 14, wherein, when the sample comprises the analyte, contacting the sample with the biosensor induces:
(a) binding of the analyte to the analyte-binding single-stranded nucleic acid molecule causing the release of the analyte-binding single-stranded nucleic acid sequence from the second nucleic acid sequence of the linear single-stranded nucleic acid molecule;
(b) an exonucleolytic digestion of the second nucleic acid sequence by the nucleic acid polymerase resulting in a mature primer nucleic sequence comprising the first nucleic acid sequence; and
(c) binding of the nucleic acid polymerase to the mature primer nucleic acid sequence to initiate rolling circle amplification (RCA) using the circular single-stranded nucleic acid molecule to produce the single-stranded nucleic acid product being monitored.

23. An analyte detection kit comprising a biosensor of claim 1 and a nucleic acid polymerase.

24. The kit of claim 23, wherein the nucleic acid polymerase is a DNA polymerase having 3′ to 5′ exonuclease activity or an RNA polymerase having 3′ to 5′ exonuclease activity.

25. (canceled)

26. The kit of claim 23, wherein the nucleic acid polymerase is ϕ29DP.

27. (canceled)

28. (canceled)

29. (canceled)


SEQUENCE LISTING (15 sequences)

SEQ ID NO: 1 - DNA, 60 bp, Artificial Sequence
  Synthetic
actgtaaccattcttgtttcgtatcattgcagaattctactaatttatctgaataccgtg

SEQ ID NO: 2 - DNA, 16 bp, Artificial Sequence
  Synthetic
gttacagtcacggtat

SEQ ID NO: 3 - DNA, 33 bp, Artificial Sequence
  Synthetic
gttacagtcacggtatatttacccaggtcagtg

SEQ ID NO: 4 - DNA, 32 bp, Artificial Sequence
  Synthetic
gttacagtcacggtatatttagccgtagcctg

SEQ ID NO: 5 - DNA, 33 bp, Artificial Sequence
  Synthetic
gttacagtcacggtatatttaggatccgacgtt

SEQ ID NO: 6 - DNA, 34 bp, Artificial Sequence
  Synthetic
  misc_feature (34)..(34): n = 3InvdT
gttacagtcacggtatatttacccaggtcagtgn

SEQ ID NO: 7 - DNA, 32 bp, Artificial Sequence
  Synthetic
cactgacctgggggagtattgcggaggaaggt

SEQ ID NO: 8 - DNA, 36 bp, Artificial Sequence
  Synthetic
  misc_feature (36)..(36): n = 3InvdT
caggctacggcacgtagagcatcaccatgatcctgn

SEQ ID NO: 9 - DNA, 36 bp, Artificial Sequence
  Synthetic
  misc_feature (36)..(36): n = 3InvdT
caggctacggcacttttttcatttaaattataattn

SEQ ID NO: 10 - DNA, 22 bp, Artificial Sequence
  Synthetic
  misc_feature (22)..(22): n = 3InvdT
aacgtcggatcccgcgtcgccn

SEQ ID NO: 11 - DNA, 21 bp, Hepatitis C virus
ggcgacgcgggatccgacgtt

SEQ ID NO: 12 - DNA, 21 bp, Hepatitis C virus
gccgatgggggatgttccgga

SEQ ID NO: 13 - DNA, 21 bp, Hepatitis C virus
gttgacgcgcaaacctacgtc

SEQ ID NO: 14 - DNA, 16 bp, Artificial Sequence
  Synthetic
gttacagtcacggtat

SEQ ID NO: 15 - DNA, 18 bp, Artificial Sequence
  Synthetic
cattgcagaattctacta